(19) United States
(12) Patent Application Publication
(10) Pub. No.: US 2003/0049305 A1
(43) Pub. Date: Mar. 13, 2003

(54) Preserved feedstuffs and process for their production

(76) Inventors: Gert-Wolfhard Von Rymon Lipinski, Schwalbach (DE); Bernd Haber, Mainz (DE); Martin Jager, Gauersheim (DE)

Correspondence Address: PROPAT L.L.C., 2912 CROSBY ROAD, CHARLOTTE, NC, 28211, US

(21) Appl. No.: 10/206,507

(22) Filed: Jul. 26, 2002

(30) Foreign Application Priority Data
Aug. 9, 2001 (DE) 101 39 162.5

(51) Int. Cl. A61K 47/00

(52) U.S. Cl. 424/439

(57) ABSTRACT

The invention relates to feedstuffs which comprise lauroylamino acid alkyl ester (LAAE)

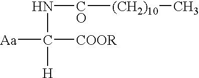

where R=unbranched or branched C₁-C₄ alkyl and

Aa=amino acid residue.

These feedstuffs are used in rearing farm animals.

The invention relates to preserved feedstuffs which prevent transfer of microorganisms to animals or significantly decrease the risk of such a transfer.

### BACKGROUND OF THE INVENTION

Feedstuffs of various types allow the growth of various microorganisms. Of these, yeasts and molds are of primary importance as sources of decay and for the formation of mycotoxins. Furthermore, they can also adversely affect digestion of the animals, and thus impair feed utilization. Preservatives such as sorbic acid, propionic acid and formic acid suppress the development of unwanted yeasts and molds in feedstuffs and thus permits disadvantageous effects of these groups of microorganisms to be avoided. However, they have only a limited action against bacteria.

In feedstuffs having a relatively high water content, bacteria are perfectly able to multiply, however, and in foods having a relatively low water content, some bacteria can survive. If such bacteria pass into the animal body there is the possibility that they multiply there and either lead to animal diseases or even transfer to the meat produced from the animals, and meat products produced therefrom.

Industrial production of feedstuffs, relatively long transport routes and relatively long storage times in animal husbandry enterprises have led to significantly increased possibilities for the growth of microorganisms in feedstuffs. Feedstuffs are thus increasingly developed into a source of contamination for the animals and the products produced therefrom.

Protection against bacterial decay using preservatives has previously not been possible satisfactorily even in the case of foods. Here, in the preservation of foods, rather more favorable starting conditions exist than with feedstuffs. It is easier in the case of food manufacture to avoid contamination of raw materials and intermediate products, since during production and storage hygienic framework conditions can be more readily maintained and thus right from the start frequently a much lower bacterial contamination is present. If satisfactory protection cannot be achieved using the permitted preservatives, generally the more complex and expensive physical preservation processes can be used, such as sterilization in the case of tinned foods, pasteurization in the case of products of limited shelf life, or deep freezing.

It is different in the case of feedstuffs, which already at the stage of raw materials, and under many conditions which are customary during production, are contaminated with considerable amounts of microorganisms and for the reasons mentioned can promote the growth of microorganisms.

The possibilities of contamination increase further under the preparation conditions which preparation takes place in practice frequently in the animal housing or in the vicinity of the animal housing, that is to say an environment having a high risk of contamination. This also applies precisely to feedstuffs having a relatively high water content which are frequently not prepared until just before feeding. In substrates which promote the growth of causative agents of decay, decay occurs more rapidly the higher the microbial count.

At the relatively high temperatures frequently prevailing in the animal housing, many microorganisms encounter fundamentally good conditions for growth. If there is a high initial level of contamination, this can increase, even with limited standing time, to microbial counts which are recognized as obvious decay. Thus in the case of preservation of feedstuffs, substantially less favorable starting conditions exist than in the case of foods. This is taken into consideration in the permitting of available preservatives for feedstuffs which, however, act preferentially against molds and yeasts and are substantially less effective against bacteria, by allowing them to be used without quantitative restriction, whereas the same substances are permitted in foods in part only at 1 g/kg and more rarely at 2 g/kg.

Obvious bacterial decay of feedstuffs can not only have disadvantageous effects on the animals, but can also even lead to contamination of meat with unsafe levels of microorganisms. This applies precisely to enterobacteria, some of which have very short generation times at relatively high temperatures. Even in the case of standing times between preparation and administration of feed with a high water content, in some circumstances a considerable increase in such bacteria can occur. However, the risk is particularly high if residues of liquid feed remain in the troughs and are consumed later by the animals.

A number of bacteria which can transfer from feedstuffs to animals are also pathogenic for humans. These include especially salmonellae, Listeria monocytogenes and certain strains of Escherichia coli, which, if they cause infections, all lead to serious disease symptoms, and not infrequently, can even cause death. In particular, salmonellaewhich are human pathogens and E. coli are encountered repeatedly in feedstuffs.

Of these, E. coli strain O157:H7 is a particular problem. It has not come to notice until recent years, but now is the subject of very close attention because it is a particular problem owing to its properties which differ highly significantly from the customary E. coli strains. Recently it has been found quite frequently in feces from ruminants, is highly resistant to environmental effects and therefore is particularly difficult to control, is further much more difficult to eliminate than other E. coli strains using customary food preservation methods, and in addition is extremely infectious. In susceptible persons, even a single intake of 100 cells is reported to lead to disease, and according to another report even 10-100 cells can lead to disease (J. Krämer: Lebensmittel-Mikrobiologie, Stuttgart 1997, pp. 50-51 and R. Steinmüller, Ernährungs-Umschau 47 (10), B 37-B 40 (2000)). The illnesses are frequently accompanied by diarrhea, but in 10-20% of ill patients, can assume a profile similar to diarrhea with bloody stools and in 5-10% of these severely ill patients, acute kidney damage can occur, which in 10% of those affected even leads to death.

To avoid contamination, it is proposed to improve hygiene, in particular during slaughter and to prevent fecal contamination (J. Krämer, loc. cit), since eradication from livestock is said not to be possible.

Protecting feedstuffs from bacteria, including those which pass into feedstuffs from contamination in animal housing is therefore a problem of great practical significance which has not been reliably solved to date, however. There is therefore a requirement firstly to eliminate contamination of feedstuffs with bacteria, in particular bacteria which are human pathogens, and secondly to exclude feedstuffs as a source of contamination of animals and foods of animal origin with such bacteria as far as possible.

There is therefore still the object of finding a simple method of protecting feedstuffs from unwanted bacteria, in particular bacteria which are human pathogens, and to exclude possible transfer of these bacteria to animals, without the risk of resistance forming or of impairing the nutritional value of the feedstuffs.

Preservatives for feedstuffs susceptible to bacteria should exhibit rapid and pronounced action against bacteria under the conditions of production and preparation of feedstuffs, because these are generally highly beneficial for the growth of bacteria. Because of their preferential action against molds and yeasts, the customary preservatives used in preservation of foods and feedstuffs, such as sorbic acid, propionic acid and formic acid, comply with this profile of requirements only in part. Although they can destroy bacteria, this is only under conditions which make the feedstuffs no longer acceptable to animals. If antibiotics are used in feedstuffs in general, and not only for therapeutic purposes, there is the risk of development of resistance which can also extend to substances which are used for therapeutic purposes in animals and humans. For this reason, the use of antibiotics in feedstuffs cannot be considered a suitable solution to the problem.

Substances having an antibacterial action which are customary in foods, cosmetics or industrial applications, cannot simply be considered suitable for use in feedstuffs. Sulfurous acid and sodium nitrite cannot be generally used in feedstuffs, for toxicological reasons alone. The substances lysozyme and nisin, which are used to a limited extent in foods, show only a very limited spectrum of activity, and, furthermore, can be degraded by enzymes in feedstuffs or microorganisms present therein. Preservatives for cosmetics or industrial applications are generally not sufficiently acceptable to animals so that they could be used on a broad basis in feedstuffs.

Further processes for increasing keeping quality which can be applied to foods, for example pasteurization, sterilization, chilling or freezing, cannot be used in feedstuffs in many cases for reasons of cost. In addition, with feedstuffs, subsequent contamination cannot be controlled, which owing to the hygiene conditions during animal feeding generally cannot be excluded.

Recently, lauroylamino acid alkyl ester (LAAE), in particular lauroylarginine ethyl ester (LAE) have been proposed as preservatives for foods (for example EP-A 0 749 960, U.S. Pat. No. 5,780,658). These substances are distinguished by high antibacterial activity, for example toward E. coli ATCC-# 9027, Staphylococcus aureus ATCC-# 8739 and toward yeasts and molds. In the body they are rapidly and completely broken down to lauric acid and the corresponding amino acids and alcohols, in the case of LAE to lauric acid, arginine and ethanol, and metabolized with production of energy. Feedstuffs having a content of LAAE, in particular LAE, and an activity, in particular, toward microorganisms which are human pathogens, are not yet known.

There is still the object of finding a simple method of protecting feedstuffs from unwanted bacteria and excluding the possible transmission to animals, without there being a risk of development of resistance and the nutritional value of the feedstuffs being impaired.

### BRIEF DESCRIPTION OF THE INVENTION

Surprisingly, it has now been found that LAAE,

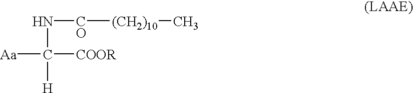

where R=unbranched or branched C₁-C₄-alkyl and

Aa=amino acid residue, in particular amino acid residue of a basic amino acid, particularly preferably an arginine, histidine, lysine or ornithine residue;

in particular lauroylarginine ethyl ester (LAE)

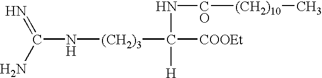

are distinguished precisely by high activity toward pathogenic bacteria.

### DETAILED DESCRIPTION OF THE INVENTION

Such pathogenic bacteria are, in foods, a potential hazard with a high risk to health, but they are much more difficult to control in feedstuffs. The activity of LAAE is surprisingly achieved even at very low concentrations, which are at the level of the preservatives otherwise used, or rather less. Even within a short time addition of LAAE leads to a significant reduction in bacterial counts, in particular of bacteria pathogenic to humans, also including the E. coli strain O157:H7 known to be particularly resistant. The occurrence of resistance to LAAE is not known. As a result, and owing to the breakdown of these compounds in the body, addition of LAAE is a particularly interesting economic and safe route for suppressing the occurrence of pathogenic bacteria in feedstuffs and as a result to minimize transfer to animal carcasses.

Constituents of LAAE which can be used are, in principle, all basic and neutral amino acids, that is to say amino acids which only contain one carboxyl group in the molecule, but preferably basic amino acids such as histidine, lysine and ornithine and very particularly preferably arginine.

The alkyl radicals are in principle freely selectable within broad limits, but are preferably unbranched or branched C₁-C₄ radicals. Very particular preference is given to the ethyl ester, also because of its more favorable physiological properties compared with the other radicals.

This means that the preferred compound for the inventive feedstuffs is lauroylarginine ethyl ester (LAE).

LAAE are prepared in a known manner, for example, by reacting amino acid alkyl esters with fatty acid chlorides (EP-A 0 749 960), or amino acids with fatty acid chlorides and subsequent esterification (GB-A 1352420).

According to the invention, feedstuffs are taken to mean complete feeds and blended feeds for farm animals including poultry and fish, and also pet food

in pieces

pulverized or finely comminuted

granulated

pelleted

and also base materials and premixes therefor. Materials which are also included are base materials and premixes for producing pasty and liquid preparations, in particular for milk replacers and starter and rearing diets.

LAAE, in the case of solid products, can be added as such or as a solution. For the preparation of solutions of LAAE, primarily 1,2-propanediol, glycerol and liquid polyethylene glycols are suitable as solvents in which LAAE is very soluble. Compounds which are also suitable for this are other polyols, for example butanediol, diethylene glycol and C₁-C₃alcohols. In addition to the pure solvents, their mixtures and mixtures with water can be used, the limited water solubility of the LAAE in practice limiting the water content to values less than 50% by weight. The content of LAAE in the solution is expediently 10 to 30% by weight, preferably 10 to 20% by weight (based on the solution).

It is possible to add LAAE to all feedstuffs which can be mechanically processed or whose surface can be sprayed with a solution. It can be added to individual feedstuffs and to compound feedstuffs. Incorporation can be carried out directly here. As an alternative, there is the possibility of adding LAAE to individual components or premixes and via these incorporating it uniformly into the feedstuff to be supplied. In the case of liquid feedstuffs or feedstuffs having a relatively high water content which have a relatively soft or pasty consistency, LAAE may also be incorporated without problem in the form of a solution, for example in propanediol.

LAAE is used in the feedstuffs expediently in minimal concentrations of 0.02 g, preferably 0.05 g, in particular 0.08 g to a maximum of 5 g, preferably 2 g, in particular 1.8 g per kg of feedstuff. If the LAAE premixes or individual constituents are added and incorporated with these into the finished feedstuff, the dosages are chosen so that these concentrations result in the finished feedstuff.

The relatively low dosages sufficient for preserving feedstuffs do not impair acceptance of the feed by the animals.

This invention is thus a very important advance in avoiding transmission of infections from animals to humans.

The invention is described in more detail by the examples below. In these examples partially desugared whey powder is used, a constituent of many feedstuffs which was set to the pH range of 6.1-6.2 which is favorable for the growth of many bacteria. The whey powder was made into a paste using aqueous buffer solution, so that together with the bacterial suspension in physiological saline a ratio of whey powder to water of approximately 2:1 resulted. LAE was added to the samples at concentrations of 200, 400 and 800 mg/kg, while a control sample received no addition. The bacterial count was determined in a conventional manner in the resultant pastes immediately after their preparation, after 24 h and after 48 h of storage at room temperature.

### EXAMPLE 1

Effect on Escherichia coli O157:H7 (approximately 6×10⁵/g)

A paste of whey powder prepared in the manner described was inoculated with approximately 6×10⁵/g of E. coli O0157:H7 EDL 933. In the control sample without LAE, after 24 h the bacterial count had decreased slightly, whereas at all concentrations of LAE a marked concentration-dependent decrease was observed, which continued further with longer storage. At an addition of 800 mg of LAE/kg, the bacterial count after 48 h was less than 10% of the value on addition.

### EXAMPLE 2

Effect on Escherichia coli O157:H7 (approximately 3×1 0³/g)

A paste of whey powder prepared in the manner described was inoculated with approximately 3×10³/g of E. coli O0157:H7 EDL 933. In the control sample without LAE, after 24 h the bacterial count had detectably decreased, but at all concentrations of LAE a much more marked concentration-dependent decrease was found which continued further with longer storage. At an addition of 800 mg of LAE/kg, the bacterial count after 48 h was less than 10% of the value on addition.

### EXAMPLE 3

Effect on Listeria monocytogenes (approximately 5×10⁵/g)

A paste of whey powder prepared in the manner described was inoculated with approximately 5×10⁵/g L. monocytogenes ATCC 13311. In the control sample without LAE, after 24 h the bacterial count had slightly decreased, whereas at all concentrations with LAE, a more marked concentration-dependent decrease was observed, which continued further with longer storage. At an addition of 800 mg of LAE/kg, the bacterial count after 48 h was approximately 15% of the value on addition.

### EXAMPLE 4

Effect on Listeria monocytogenes (approximately 2×10³/g)

A paste of whey powder prepared in the manner described was inoculated with approximately 2×10³/g L. monocytogenes ATCC 13311. In the control sample without LAE, after 24 h the bacterial count had decreased slightly, whereas at all concentrations with LAE, a more marked concentration-dependent decrease was observed, which continued further with longer storage. At an addition of 800 mg of LAE/kg, the bacterial count after 48 h was less than 20% of the value on addition.

### EXAMPLE 5

Effect on Salmonella typhimurium (approximately 3×10⁴/g)

A paste of whey powder prepared in the manner described was inoculated with approximately 3×10⁴/g S. typhimurium NCTC 10527. In the control sample without LAE, after 24 h the bacterial count had detectably decreased, but at all concentrations of LAE, a much more marked concentration-dependent decrease was found which continued further with longer storage. At an addition of 800 mg of LAE/kg, the test bacteria were no longer detectable after 48 h.

1. A feedstuff which has a content of lauroylamino acid alkyl ester (LAAE) 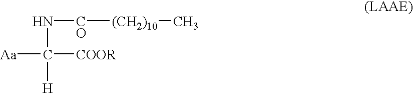
where R=unbranched or branched C₁-C₄ alkyl and
Aa=amino acid residue.

2. The feedstuff as claimed in claim 1, wherein the lauroylamino acid alkyl ester is lauroylarginine ethyl ester (LAE)

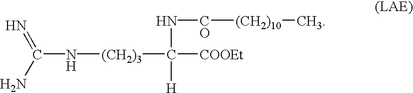

3. The feedstuff as claimed in claim 1, wherein the LAAE content is 0.02 to 5 g per kilogram of feedstuff.

4. The feedstuff as claimed in claim 1, wherein solid LAAE is incorporated.

5. The feedstuff as claimed in claim 1, wherein LAAE is incorporated or applied in dissolved form.

6. The feedstuff as claimed in claim 1, wherein it comprises a solution of LAAE.

7. The feedstuff as claimed in claim 6, wherein 1,2-propanediol, glycerol, liquid polyethylene glycols or their mixtures or mixtures of these substances with water are used as solvent.

8. The feedstuff as claimed in claim 6, wherein the LAAE content of the solution is 10 to 30% by weight.

9. The method of making a feedstuff for rearing farm animals which comprises incorporating LAAE into a feedstuff.

10. The method as claimed in claim 9, wherein the farm animals are cattle, sheep, pigs, poultry or fish.
